(19) United States
(12) Patent Application Publication
(10) Pub. No.: US 2017/0190670 A1
(43) Pub. Date: Jul. 6, 2017

(54) IMPROVED PROCESS FOR THE PREPARATION OF ENZALUTAMIDE

(71) Applicant: SHILPA MEDICARE LIMITED, Raichur Karnataka (IN)

(72) Inventors: SANJAY HIRPARA, RAICHUR (IN); VIMAL KUMAR SHRAWAT, RAICHUR (IN); RAJU POTHURAJU, RAICHUR (IN); CHANDRESH KUMAR TRIPATHI, RAICHUR (IN); CHATURVEDI AKSHAYKANT, RAICHUR (IN)

(73) Assignee: SHILPA MEDICARE LIMITED, RAICHUR, OT (IN)

(21) Appl. No.: 15/325,085

(22) PCT Filed: Jul. 6, 2015
(86) PCT No.: PCT/IB2015/055087

§ 371 Date: Jan. 10, 2017

(30) Foreign Application Priority Data
Jul. 11, 2014 (IN) 3447/CHE/2014
Oct. 11, 2014 (IN) 5111/CHE/2014

(51) Int. Cl. C07D 233/86

(52) U.S. Cl. CPC C07D 233/86; C07B 2200/13

(57) ABSTRACT

The present application relates to an improved process for preparation of Enzalutamide (I).

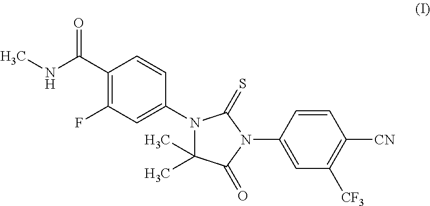 [SMILES: CNC(=O)c1ccc(N2C(=S)N(c3ccc(C#N)c(C(F)(F)F)c3)C(=O)C2(C)C)cc1F]

The present application also relates to an improved process for the preparation of substantially pure Enzalutamide (I) having purity of greater than 99.5%.

The present application also relates to a novel process for the preparation of Enzalutamide intermediate useful in the industrially viable synthesis of Enzalutamide.

### FIELD OF THE INVENTION

The present invention relates to an improved process for preparation of Enzalutamide of Formula (I).

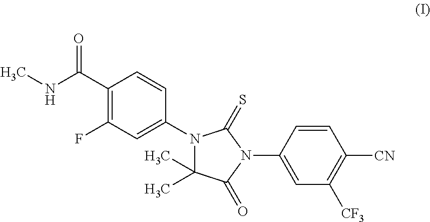 [SMILES: CNC(=O)c1ccc(N2C(=S)N(c3ccc(C#N)c(C(F)(F)F)c3)C(=O)C2(C)C)cc1F]

### BACKGROUND OF THE INVENTION

Enzalutamide is chemical known as 4-{3-[4-cyano-3-(trifluoromethyl)phenyl]-5,5-dimethyl-4-oxo-2-sulfanylide-neimidazolidin-1-yl}-2-fluoro-N-methylbenzamide. The structural formula of Enzalutamide is as described in Formula (I)

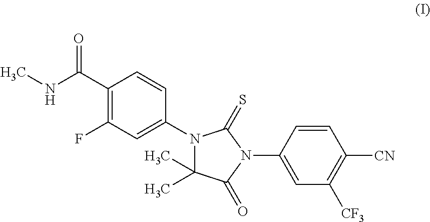 [SMILES: CNC(=O)c1ccc(N2C(=S)N(c3ccc(C#N)c(C(F)(F)F)c3)C(=O)C2(C)C)cc1F]

Enzalutamide is an androgen receptor inhibitor that acts on different steps in the androgen receptor signaling pathway. Enzalutamide has been shown to competitively inhibit androgen binding to androgen receptors and inhibit androgen receptor nuclear translocation and interaction with DNA. A major metabolite, N-desmethyl Enzalutamide, exhibited similar in-vitro activity to Enzalutamide. Enzalutamide decreased proliferation and induced cell death of prostate cancer cells in-vitro, and decreased tumor volume in a mouse prostate cancer xenograft model. Prostate cancer is the most commonly diagnosed cancer among men in the United States, other than skin cancer. Prostate cancer is thus the second-leading cause of cancer death in men in the United States, after lung cancer.

Enzalutamide is a white crystalline non-hygroscopic solid with the empirical formula C₂₁H₁₆F₄N₄O₂S and a molecular weight of 464.44. It is practically insoluble in water and freely soluble in NMP and acetonitrile, sparingly soluble in absolute ethanol.

Enzalutamide is achiral, therefore no stereoisomerism is observed. The pure drug substance melts at 201° C. Enzalutamide is marketed under the brand name Xtandi® as an oral capsule. Enzalutamide is specifically disclosed in U.S. Pat. No. 7,709,517. This patent discloses a process for the preparation of Enzalutamide starting from 2-fluoro-4-nitrotoluene, which is as demonstrated below:

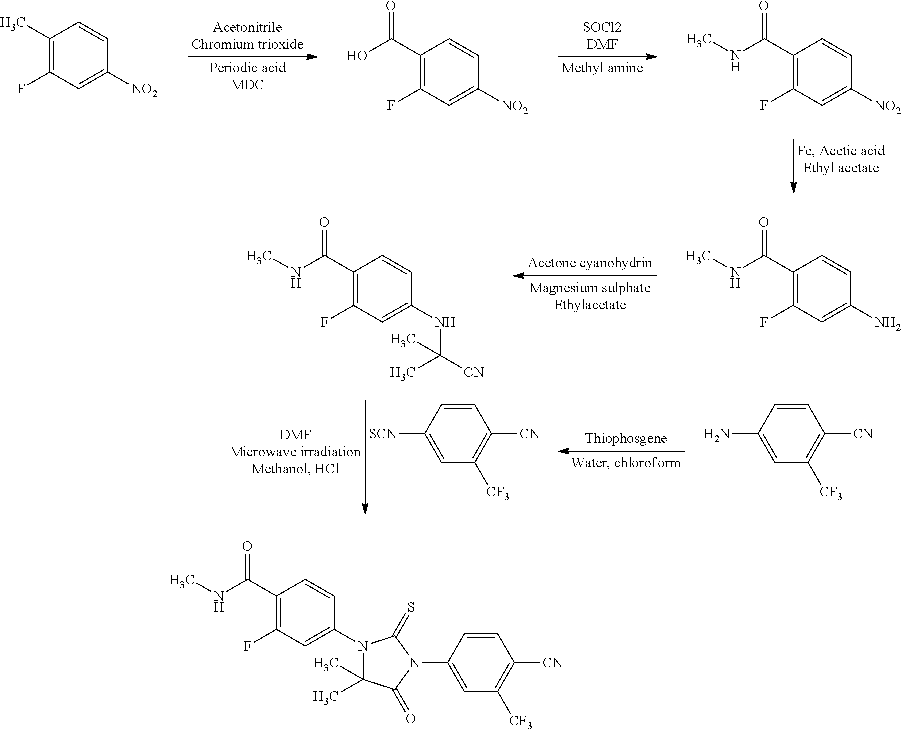

This patent discloses that the concentrated residue of Enzalutamide is purified by SiO₂ column chromatography using dichloromethane and acetone as mobile phase solvents to yield colorless crystals of Enzalutamide.

US 2013/0190507 disclose different processes for the preparation of Enzalutamide starting from N-methyl-2-fluoro-4-bromobenzamide, which is demonstrated below:

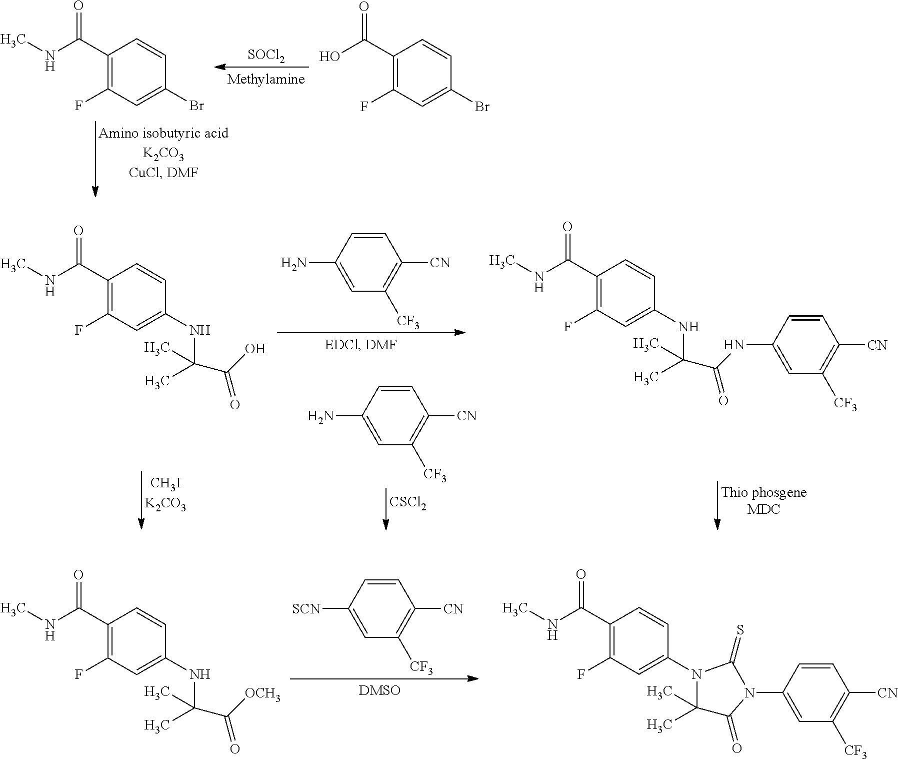

This patent publication not disclosed any separate purification of Enzalutamide. However, the use of 2-acetyl cyclohexanone as a ligand in presence of DMF in the coupling of 4-bromo-2-fluoro-N-methyl benzamide with 2-amino isobutyric acid leads to the formation of unwanted reactions.

WO 2014/041487 discloses two crystalline forms of Enzalutamide namely R1 and R2. This patent discloses a process for the preparation of crystalline form R1 of Enzalutamide comprising

a) providing a solution of Enzalutamide in a solvent; and

b) isolating crystalline Enzalutamide Form R1.

wherein disclosed solvents for preparing Form-R1 includes acetonitrile, ethylacetate, n-butyl acetate, MIBK, Xylene, N,N-DMF, NPM, THE etc.

As described above U.S. Pat. No. 7,709,517 disclose that the concentrated residue of Enzalutamide is purified by SiO₂ column chromatography using dichloromethane and acetone as mobile phase solvents to yield colorless crystals of Enzalutamide.

The general column chromatography process involves the following steps:
a) plug a Pasteur pipet with a small amount of cotton
b) add dry/wet Silica gel (SiO₂) as adsorbent.
c) pre-elute the column by adding the solvent to flow slowly down the column;
d) load the sample onto the silica gel column
e) add fresh eluting solvent (mixture of dichloromethane:acetone in a ratio of 95:5 as disclosed in US '517) to the top and start the elution process.
f) separate bands are observed and these fractions are collected separately; and
g) analyze the fractions

The fractions are collected in the form of solution containing the product. The product is isolated by evaporating the solvent. A reproduction of the process by the present inventors especially step a) to g) has demonstrated that above resulted in the crystalline material possessing PXRPD resembling to form-R1. It was also observed that cumbersome process of column purification is desirable in order to remove the significant levels of impurities formed in the process. However, it was also observed that said crystalline form was found thermodynamically stable and reproducible by other solvent systems like acetonitrile, ethyl acetate, MIBK, xylene, DMF etc as disclosed in WO '487 for the preparation of crystalline R1 of Enzalutamide.

The present inventors has repeated the above process and found the following disadvantages:
In most of the patent literature, N,N′-Dimethylformamide is used as a solvent in coupling stage of 4-bromo-2-fluoro-N-methyl benzamide with 2-amino isobutyric acid, which results in poor yields due to decomposition of product.
Unwanted side reactions are observed during the formation of Enzalutamide, due to the use of microwave irradiation at higher temperature. Further, the use of microwave irradiation at high temperature is hazardous, and is industrially not feasible.
Condensation of 2-(3-Fluoro-4-methylcarbamoyl-phenylamino)-2-methyl-propionic acid methylester with 2-(trifluoromethyl)-4-isothiocyanato benzonitrile involves the use of higher temperature i.e., around 83-84° C., Use of 2-acetyl cyclohexanone as a ligand in DMF in the coupling reactions leads to the formation of unwanted reactions.

In view of the above and to overcome the prior-art problems the present inventors have now developed an improved user friendly process for the preparation of Enzalutamide, using minimal steps, which is industrially feasible process, with the use of industrial friendly solvents, which neither involves tedious work up nor any hazardous handling steps.

### Objective of the Invention

The main objective of the invention is to provide an improved process for the preparation of Enzalutamide.

Yet another objective of the invention is to provide an improved process for the preparation of substantially pure Enzalutamide having purity of greater than 99.5%.

Yet another objective of the invention is to provide an improved process for the preparation of Enzalutamide intermediate.

Yet another objective of the invention is to provide substantially pure Enzalutamide having a purity of greater than 99.5%.

Yet another objective of the invention is to provide substantially pure crystalline Enzalutamide having XRPD pattern comprising at least 7 characteristic peaks possessing peaks selected from 6.5, 9.8, 13.1, 15.8, 16.0, 16.7, 18.9, 19.5, 19.7, 21.2, 22.6, 25.5±0.2°2θ

### SUMMARY OF THE INVENTION

The present invention relates to an improved process for the preparation of Enzalutamide of Formula (I)

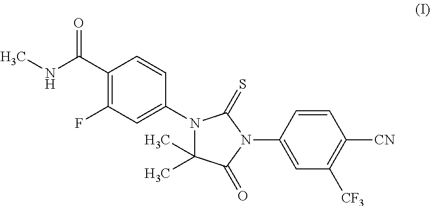 [SMILES: CNC(=O)c1ccc(N2C(=S)N(c3ccc(C#N)c(C(F)(F)F)c3)C(=O)C2(C)C)cc1F]

comprising the steps of:
a) preparation of 4-bromo-2-fluoro-N-methyl benzamide (III);
   i. reacting 4-bromo-2-fluorobenzoic acid of Formula (II)

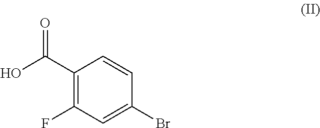 [SMILES: O=C(O)c1ccc(Br)cc1F]

with a chlorinating agent in presence of a solvent-1, followed by condensation with Methylamine in presence of a solvent-2; or

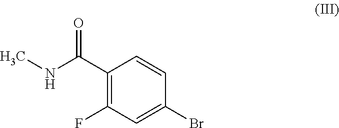

ii. reacting 4-bromo-2-fluorobenzoic acid of Formula II

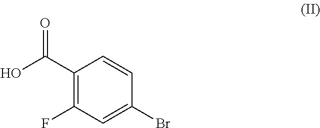 [SMILES: O=C(O)c1ccc(Br)cc1F]

with a chlorinating agent in presence of a solvent; in the ratio between 3-8 v/w times, followed by condensation with Methylamine
b) preparing 2-(3-Fluoro-4-methylcarbamoyl-phenylamino)-2-methyl-propionic acid (IV)
   i. reacting the compound of formula (III) with 2-amino isobutyric acid in presence of amino acid and a solvent, where in the solvent is selected from a mixture of ether and an organic solvent in a ratio of 1:9 to 9:1; or

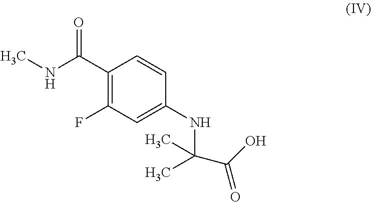 [SMILES: CNC(=O)c1ccc(NC(C)(C)C(=O)O)cc1F]

ii. reacting the compound of formula (III) with 2-amino isobutyric acid in presence of ligand and a solvent;
c) reacting the compound of Formula IV with 2-(trifluoromethyl)-4-isothiocyanato benzonitrile (V) in presence of base and a solvent to provide Enzalutamide (I); and

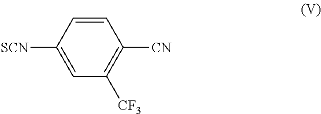

d) purifying the compound obtained in step c) further comprises of
   i. providing a solution of Enzalutamide obtained in step-c) using a solvent selected from alcohol (C1-4) or Ketones (C3-6) or organic solvents (C1-8 alkanes, dimethyl formamide) or halogenated organic solvents (Methylene dichloride, Ethylene dichloride) or Ethers (Methyl tertiary butyl ether, tetrahydrofuran, Di-isopropyl ether) or sulphoxides (dimethyl sulphoxide), water or mixtures thereof;
   ii. acidifying the solution using an acid selected from organic/inorganic acid not limited to formic acid, citric acid, acetic acid, Hydrochloric acid; and
   iii. isolating the substantially pure Enzalutamide having a purity of greater than 99.5%.
      In another aspect of the present invention relates to process for the preparation of Enzalutamide (I) comprising the steps of:
   a) reacting 4-bromo-2-fluorobenzoic acid of Formula (II) with chlorinating agent in presence of a solvent, followed by condensation with Methylamine to yield 4-bromo-2-fluoro-N-methyl benzamide of Formula (III)

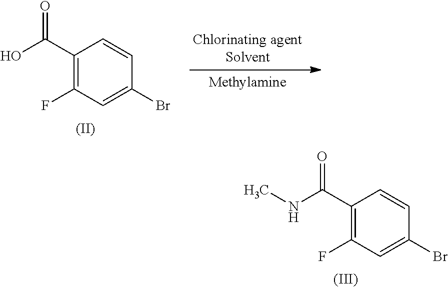

b) reacting the compound of formula (III) with 2-amino isobutyric acid in presence of ligand and a solvent to give 2-(3-Fluoro-4-methylcarbamoyl-phenylamino)-2-methyl-propionic acid of Formula IV

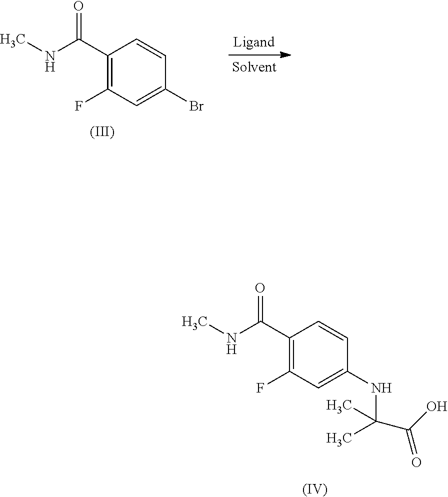

c) converting 2-(3-Fluoro-4-methylcarbamoyl-phenylamino)-2-methyl-propionic acid of Formula IV to Enzalutamide (I)

In another aspect the present invention relates to substantially pure Enzalutamide having a purity of greater than 99.5%.

In another aspect of the present invention relates to substantially pure crystalline Enzalutamide having XRPD pattern comprising at least 7 characteristic peaks possessing peaks selected from 6.5, 9.8, 13.1, 15.8, 16.0, 16.7, 18.9, 19.5, 19.7, 21.2, 22.6, 25.5±0.2°2θ designated as Form SEZ.

### BRIEF DESCRIPTION OF THE DRAWINGS

FIG. 1: PXRD pattern of crystalline Enzalutamide obtained as per the present invention.

### DETAILED DESCRIPTION OF THE INVENTION

The present invention relates to an improved process for the preparation of Enzalutamide of Formula (I), comprising the steps of reacting 4-bromo-2-fluorobenzoic acid of Formula (II) with a chlorinating agent selected from Oxalyl chloride, Thionyl chloride, PCl₃, PCl₅, POCl₃ at a temperature ranging from 25-55° C. and in presence of a solvent-1 selected from solvent selected from halogenated hydrocarbons such as methylene dichloride, ethylene dichloride, chloroform; esters such as ethyl acetate, isopropyl acetate, isobutyl acetate, methyl acetate; sulfoxides such as dimethylsulfoxide; aromatic hydrocarbons such as toluene, xylene; ketones such as acetone, methyl ethyl ketone, methyl isobutyl ketone; water or mixtures thereof. The reaction mixture was stirred for 2 to 3 hours to yield a solution containing chlorinated product. The solvent is distilled off to yield acid chloride product in the form of residue.

The acid chloride was chlorinating agent selected from Oxalyl chloride, Thionyl chloride, PCl₃, PCl₅, POCl₃; in presence of a solvent selected from halogenated hydrocarbons such as methylene dichloride, ethylene dichloride, chloroform; esters such as ethyl acetate, isopropyl acetate, isobutyl acetate, methyl acetate or mixtures thereof; in the ratio between 3-8 v/w times; at a temperature ranging from 20-55° C.; to obtain acid chloride as a residual mass.

The present inventors found that the use of oxalyl chloride as a chlorinating agent completes the reaction at a low temperature ranging from 25-30° C., which is industrially feasible, cost effective and avoids unwanted reactions, which minimized the formation of impurity. After completion of the reaction, the removal of oxalyl chloride is modest and does not require any cumbersome workup.

However, the prior art patents includes the use of thionyl chloride as a chlorinating agent moves the reaction at reflux temperature i.e., at 60-110° C., which is tedious and cumbersome.

To the above obtained residue Methylamine was added at a temperature ranging from 10-15° C. in presence of a solvent-2 selected from ethers such as Methyl tert-butyl ether, Tetrahydrofuran, Methoxyethane, Di-tert-butyl ether, Diethyl ether, Di-ethylene glycol diethyl ether, Diglyme, Di-isopropyl ether, Dimethoxymethane, 1,4-Dioxane, 1,3-dioxane, 1,2-dimethoxy ethane, Ethyl tert-butyl ether, 2-Methyl tetrahydrofuran, Morpholine; Glycol ethers such as 2-Butoxyethanol, Diglyme, Dimethoxyethane, 2-Ethoxy ethanol, 2-(2-Ethoxyethoxy)ethanol, 2-Methoxyethanol, 2-(2-Methoxyethoxy) ethanol, Octaethylene glycol monododecyl ether, Pentaethylene glycol monododecyl ether, Phenoxyethanol, Propylene glycol methyl ether acetate, Tetraethylene glycol dimethyl ether, Triethylene glycol, Triethylene glycol dimethyl ether; water or mixtures thereof. The obtained reaction mixture was stirred at a temperature ranging from 25-40° C. for 1-2 hours. To the obtained reaction mass water was added and stirred for 30 min to 1 hour. Separated the organic layer and the aqueous layer was extracted twice with ester solvent. The total organic layer was washed with sodium bicarbonate solution, followed by citric acid solution. The organic layer was concentrated to yield 4-bromo-2-fluoro-N-methyl benzamide (III)

The present inventors surprisingly found that the use of ether solvent or ester solvent in the condensation step leads to the formation of pure compound of Formula (III), which is devoid of other process related impurities. Further, the prior-art process utilizes ester solvent in the methyl amine condensation step. However, the acid chloride obtained is not much freely soluble in ester solvent and the reaction is incomplete leads to the formation of lower yields as well unwanted by-products.

The obtained compound of formula (III) is reacted with 2-amino iso butyric acid in presence of ligand selected from 2-acetyl cyclohexanone, N,N-dimethyl glycine.HCl, amino acid selected from cyclic amino acid such as Proline, L-Proline, D-Proline, Hydroxyproline, Pseudoproline, 1-Aminocyclopropane-1-carboxylic acid, Azetidine-2-carboxylic acid; in a solvent selected from alcohols, such as ethanol, ethylene glycols, n-butanol, isopropanol; ethers such as tetrahydrofuran, 1,4-dioxane, 1,3-dioxane, 1,2-dimethoxy ethane; aprotic polar solvents like acetonitrile, dimethylformamide, dimethylsulfoxide or mixtures thereof in a ratio of 1:9 to 9:1 at a temperature ranging from 60 C.° to 130° C. for 20-30 hours to provide 2-(3-Fluoro-4-methylcarbamoyl-phenylamino)-2-methyl-propionic acid of Formula IV.

The present inventors found that the use of dimethyl formamide as a solvent in coupling stage of 4-bromo-2-fluoro-N-methyl benzamide with 2-amino isobutyric acid yield lower yield due to the decomposition of product. The present inventors found that the decomposition of the product is due to the distillation of solvent at high temperature. Further, the present inventors found that the reaction is incomplete as the final product contains 20-30% of the compound of formula (III) leads to formation of lower yields. Further, the prior art patents includes the use of thiophosgene, which is highly hazardous and is very laborious for handling at large scale.

In view of this, the present inventors developed an improved process for the preparation of Enzalutamide, by using industrial friendly solvents and reagents, which leads in the formation of good yield with high purity.

The present inventors surprisingly found that the compound of formula (III) is freely soluble in the solvent mixture containing ether and organic solvent yields in good yield as well as pure product, which is free of process related impurities.

Another aspect of the present invention is amino acid as a ligand. The present inventors surprisingly found that the use of amino acid as a ligand yield in the pure compound of Formula (IV), which is devoid of process related impurities. The present inventors found that the use of amino acid as a ligand, which is an industrial friendly reagent leads in the formation of good yield with high purity.

The use of amino acid moves the reaction more freely compare to the ligands used in prior art such as 2-acetyl cyclohexanone. This is due to the high solubility nature of amino acids in water. Specifically, the use of L-proline in this reactions leads to highly pure product, which is devoid of process related impurities. Further, the use of amino acids in large scale is highly cost effective and environmental friendly.

The present inventors found that the use of 2-acetyl cyclohexanone/acetone cynohydrin as a ligand will not support the reaction as much as supported by L-proline, this is may be due to mis-match of the Redox potential. Further, the lone pair of electrons on the nitrogen atom in the L-proline may be useful to form a ligand complex with Copper and enhances the rate of the reaction.

The obtained compound of Formula IV is reacted with 2-(trifluoromethyl)-4-isothiocyanato benzonitrile of Formula (V) in presence of base selected from inorganic or organic bases, such as triethylamine, diisoproylethylamine, tributyl amine, N,N-dimethyl aniline, pyridine, N-methylmorpholine, DBN, DBU; in a solvent selected from chlorinated solvent such as methylene dichloride, chloroform; ketone solvents such as acetone, methyl isobutyl ketone; acetonitrile or mixtures thereof; at a temperature ranging from 25-30° C. for about 20 hours. The reaction mass was filtered and distilled off to give Enzalutamide in the form of a residue.

The obtained residue was purified by treating the residue with a suitable solvent selected from, but are not limited to: alcohols, such as C2-C6 alcohols like ethanol, 1-propanol, 2-propanol (isopropyl alcohol), 1-butanol, 2-butanol, t-butyl alcohol; or nitriles, such as acetonitrile or propionitrile; amides such as N,N-dimethylformamide, N,N-dimethylacetamide, N-methyl-2-pyrrolidone; sulfoxides such as dimethylsulfoxide; halogenated hydrocarbons such as dichloromethane; aromatic hydrocarbons such as toluene, xylene; esters such as ethyl acetate, n-propyl acetate, n-butyl acetate, isopropyl acetate, isobutyl acetate, t-butyl acetate; ethers such as diethyl ether, diisopropyl ether, methyl t-butyl ether, tetrahydrofuran, 1,4-dioxane, 2-methoxyethanol, anisole; ketones such as acetone, ethyl methyl ketone, diethyl ketone, methyl isobutyl ketone; organic solvents such as dimethyl formamide, n-hexane, n-heptane, cyclohexane, cycloheptane; water; or any mixtures of one or more of these solvents; at a temperature ranging from 25-60° C. for about 30 minutes to 2 hours to yield pure Enzalutamide. The purification is repeated using different solvents to obtain the desired purity.

Purification of Enzalutamide further comprises of providing a solution of Enzalutamide using a solvent selected from alcohol (C1-4) or Ketones (C3-6) or organic solvents (C1-8 alkanes, dimethyl formamide) or halogenated organic solvents (Methylene dichloride, Ethylene dichloride) or Ethers (Methyl tertiary butyl ether, tetrahydrofuran, Di-isopropyl ether) or sulphoxides (dimethyl sulphoxide), water or mixtures thereof; acidifying the solution using an acid selected from organic/inorganic acid not limited to formic acid, citric acid, acetic acid, Hydrochloric acid; and isolating the substantially pure Enzalutamide having a purity of greater than 99.5%.

Purification of Enzalutamide further comprises of providing a solution of Enzalutamide using a solvent selected from alcohol such as ethanol, 1-propanol, 2-propanol (isopropyl alcohol), 1-butanol, 2-butanol, t-butyl alcohol; organic solvents such as dimethyl formamide, n-hexane, n-heptane, cyclohexane, cycloheptane; ketones such as acetone, methyl ethyl ketone, methyl isobutyl ketone; halogenated hydrocarbons such as dichloromethane; aromatic hydrocarbons such as toluene, xylene; esters such as ethyl acetate, n-propyl acetate, n-butyl acetate, isopropyl acetate, isobutyl acetate, t-butyl acetate; ethers such as diethyl ether, diisopropyl ether, methyl t-butyl ether, tetrahydrofuran, 1,4-dioxane, 2-methoxyethanol, anisole; sulfoxides such as dimethyl sulfoxide; water or mixtures thereof; acidifying the solution using an acid selected from organic/inorganic acid not limited to formic acid, oxalic acid, Malic acid, citric acid, acetic acid, Hydrochloric acid, sulphuric acid, nitric acid, boric acid, perchloric acid, phosphoric acid or mixtures thereof. The obtained reaction mixture was stirred for 30 minutes to 3 hours at a temperature ranging from 25-30° C. Filtered the material and washed with corresponding solvent or water. The obtained solid material was dried to yield substantially pure Enzalutamide having a purity of greater than 99.5%.

In another embodiment of the invention is purifying the Enzalutamide without using any solvent by repeating the same purification process as disclosed above. The purification further involves the isolation of solid Enzalutamide and washing of the solid Enzalutamide to obtain pure Enzalutamide, which is devoid of process related impurities and to meet ICH guidelines.

The Enzalutamide obtained as per the present invention is highly pure and having a purity of greater than 99.5%. This purity is achieved due to the formation of pure intermediates, which are devoid of process related impurities.

In view of this, the present inventors developed an improved process for the preparation of Enzalutamide, by using industrial friendly solvents and reagents, which leads in the formation of good yield with high purity.

The present inventors developed a process for the preparation of Enzalutamide, wherein the reaction course is extremely smooth and achievable at room temperature conditions of 25-30° C., which is not only industrially feasible but also cost effective and provide pure materials/intermediates. Exceptional advantage of the said process of the present invention was that—it does not require cumbersome process such as use of microwave irradiation at an elevated temperatures i.e., around 83-84° C. and prolonged hours e.g. as disclosed in US '517 example 56 such microwave dependent reaction was carried out up to exceeding 72 hours and resulting in poor yields and exceptional levels of impurity formation.

To overcome these serious industrially non-viable process concerns as cited above—especially use of microwave irradiation based reaction, the present inventors aimed for a process, which is not only industrially upscale process but also cost effective and least time consuming. The inventors in the present invention found that the use of base in the condensation step makes the reaction to move smoothly at ordinary lower temperatures i.e. at about 25-30° C., which was found to help in avoiding the formation of large number impurities due to unwanted parallel reactions and resulting in recovering purer material.

Drying may be also be performed by any conventional process not limited to spray drying or distillation to remove the solvent. Drying may be performed under reduced pressure conditions also. Reduced pressure conditions may be suitably utilized by person skilled in the art in order to obtain the dried material. The drying may be performed at a temperature ranging from 50-60° C. for time ranging from 12 to 16 hrs depending upon the physical attributes of the end product obtained i.e. Pure Enzalutamide.

In another embodiment pure Enzalutamide obtained above is substantially pure and having a purity of greater than 99.5%.

In another embodiment pure Enzalutamide obtained above is substantially pure and having a purity of greater than 99.6%.

In another embodiment pure Enzalutamide obtained above is substantially pure and having a purity of greater than 99.7%.

In another embodiment pure Enzalutamide obtained above is substantially pure and having a purity of greater than 99.8%.

In another embodiment pure Enzalutamide obtained above is substantially pure and having a purity of greater than 99.9%.

In another embodiment substantially pure crystalline Enzalutamide obtained by the present process is having XRPD pattern comprising at least 7 characteristic peaks possessing peaks selected from 6.5, 9.8, 13.1, 15.8, 16.0, 16.7, 18.9, 19.5, 19.7, 21.2, 22.6, 25.5±0.2°2θ Substantially pure crystalline Enzalutamide obtained is further characterized by XRPD peaks at 12.3, 13.5, 14.3, 15.0, 17.4, 21.8, 24.4±0.2°2θ

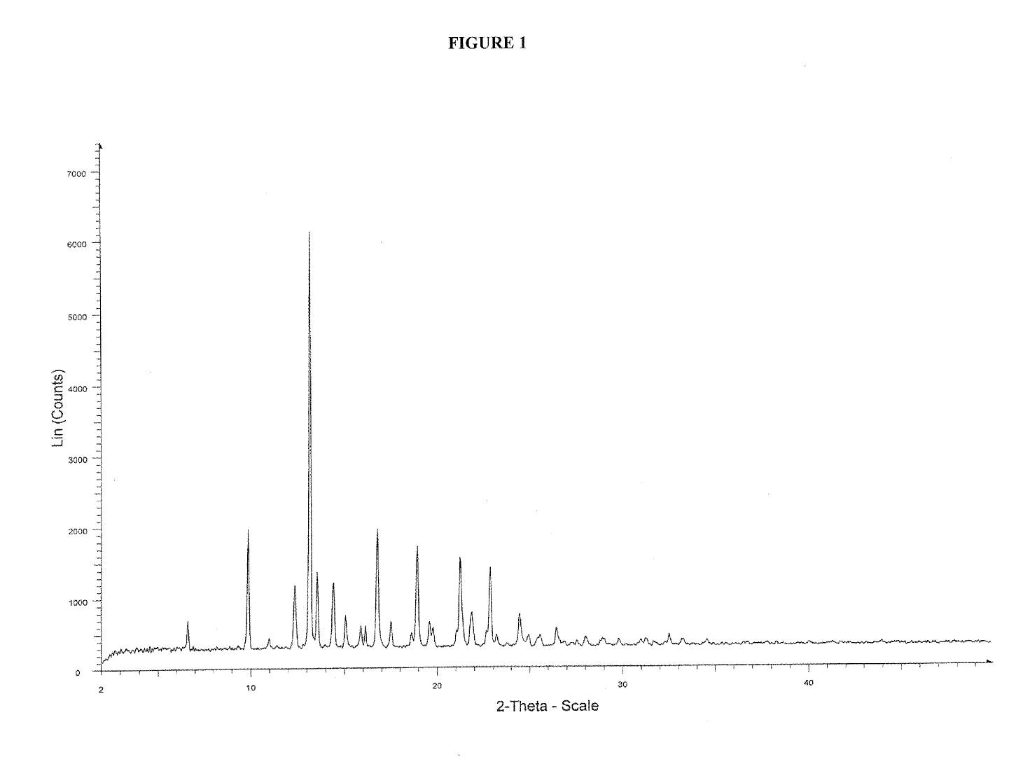

Substantially pure crystalline Enzalutamide obtained by the process of the present invention is characterized by X-ray powder diffraction pattern substantially according to FIG. 1

Substantially pure crystalline Enzalutamide obtained is further characterized by XRPD peaks at 12.3, 13.5, 14.3, 15.0, 17.4, 21.8, 24.4±0.2°2θ

The process related impurities that appear in the impurity profile of the Enzalutamide may be substantially removed by the process of the present invention resulting in the formation of highly pure material. The process of the present invention is as summarized in the Scheme-I as represented below:

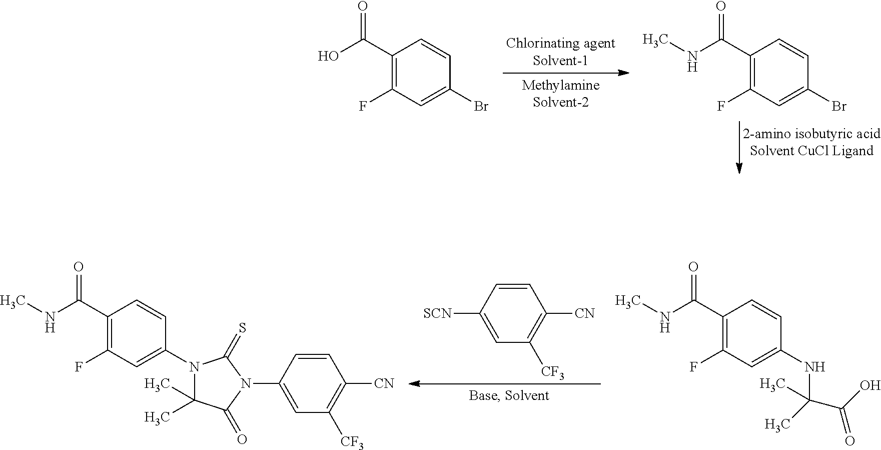

Scheme-I: Process for Preparation of Enzalutamide of the Present Invention

In another embodiment, the Enzalutamide obtained by the processes of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Enzalutamide obtained as per the present application process—include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions derived from Enzalutamide of the present application may also comprise to include the pharmaceutically acceptable carrier used for the preparation of solid dispersion, wherever utilized in the desired dosage form preparation.

The following examples illustrate the nature of the invention and are provided for illustrative purposes only and should not be construed to limit the scope of the invention.

### EXAMPLES

### Example 1

### Preparation of 4-bromo-2-fluoro-N-methyl benzamide (III)

N,N-Dimethylformamide (0.5 mL, 0.006 mol) was added to the suspension of 4-bromo-2-fluorobenzoic acid (10.0 g, 0.045 mol) in dichloromethane (70 mL) at 10 to 15° C. To the reaction mixture oxalyl chloride (8.0 mL, 0.093 mol) was added drop wise and stirred at 25-30° C. for 2 to 3 hrs. Distill off the solvents to get residue. Methyl tertiary butyl ether (100 mL) was added to the residue and cooled to 10 to 15° C. To the obtained reaction mixture aqueous methyl amine (40%) was added drop wise at a pH around 8 to 9. The reaction mixtures was stirred at 25-30° C. for 30 min to 1 hr. Add DM water and stir for 30 min and separate the organic layer. The aqueous layer was extracted twice with Dichloromethane (2×100 ml) and combined organic layer washed with 5% citric acid solution (100 mL). The organic layer was washed with 100 ml of 5% NaHCO₃ solution followed by 200 ml of DM water wash. The organic layer was concentrated to obtain title product as off-white solid.

Yield: 7.6 gm

### Example 2

### Preparation of 2-(3-Fluoro-4-methylcarbamoyl-phenylamino)-2-methyl-propionic acid (IV)

4-bromo-2-fluoro-N-methyl benzamide (48.0 gm, 0.21 mol) and K₂CO₃ (52.8 gm, 0.38 mol) was charged in to reaction flask containing DMF (67.2 mL), 1,4-dioxane (268 mL) and DM water (8.6 mL). The reaction mixture was purged with nitrogen gas for 20 min and then charged CuCl (6.24 gm, 0.06 mol), L-proline (9.6 gm, 0.08 mol) and 2-aminoisobutyric acid (31.6 gm, 0.31 mol). Continue purging nitrogen gas for 20 min followed by refluxed for 24 hrs. Solvent is distilled off completely from the obtained reaction mixture under vacuum at 70° C. The reaction mass was cooled to 10 to 15° C. and then charged DM water (20 mL). The pH of the reaction mass adjusted to 3-5 with 1 M citric acid solution to obtain the title product.

Yield: 26.0 gm

Chromatographic Purity (By HPLC): 95.38%

### Example 3

### Preparation of Enzalutamide (I)

2-(trifluoromethyl)-4-isothiocyanato benzonitrile (100.0 gm, 0.44 mol), 2-(3-Fluoro-4-methylcarbamoyl-phenylamino)-2-methyl-propionic acid (50.0 gm, 0.20 mol) were charged in to the reaction flask containing Dichloromethane (500 mL), triethylamine (50.0 mL, 0.36 mol) at 25-30° C. and stirred for 20 hrs. The reaction mass was filtered and filtrate was distilled off to get residue. The obtained residue Methanol (500 mL) was added and heated to reflux. To the obtained reaction mass DM water (250 mL) was added at reflux and slowly allowed to cool to room temperature to get solid. Filter the solid Ethyl acetate (500 mL) was added at 25-30° C. and stirred for 3 hours. Filter the material and washed with ethyl acetate. The obtained filtrate is passed through celite bed and washed with ethyl acetate. To the obtained filtrate n-Heptane (1000 mL) was added and stirred for 3 hrs. Filter the solid and washed n-heptane. To the obtained solid Isopropyl alcohol (720 mL) was added and refluxed to get clear solution. Slowly cool the reaction mixture to 25-30° C. to obtain the title product.

Yield: 20.0 gm

Chromatographic Purity (By HPLC): 99.18%

Purification may be further carried out using the same solvent/s and recrystallized product obtained resulted in the purity exceeding 99.5% (by HPLC).

### Example 4

Purification of Enzalutamide

Enzalutamide (34 gm) was purified through column chromatography using 60-120 mesh silica gel using Ethyl acetate, Acetone and heptane as solvents. The solvent fractions were collected and concentrated to obtain pure Enzalutamide.

Yield: 32.0 gm

Chromatographic Purity (By HPLC): 99.82%

### Example 5

Purification of Enzalutamide

Enzalutamide (24 gm) was charged in to the reaction flask containing Isopropanol (182.0 ml) and dimethyl sulfoxide (9.6 ml) and heated to 80-90° C. The obtained reaction mass stirred for 10 to 15 minutes and filtered through celite bed. The filtrate was slowly cooled to 25-30° C. to obtain solid. The obtained solid was washed with isopropanol. Suck dried the material to yield pure Enzalutamide. The obtained Enzalutamide was charged in to the reaction flask containing hydrochloric acid (200 ml) and stirred for 2 hours at 25-30° C. Filter the material and washed with water. The obtained solid material was dried under vacuum at a temperature ranging from 50-60° C. DM for 4 to 5 hours to yield the pure Enzalutamide

Yield: 18.0 gm

Chromatographic Purity (By HPLC): 99.86%

### Example 6

Purification of Enzalutamide

Enzalutamide (18 gm) was charged in to the reaction flask containing hydrochloric acid (200 ml) and stirred for 2 hours at 25-30° C. Filter the material and washed with water. Suck dried the material to yield pure Enzalutamide.

Yield: 17.0 gm

Chromatographic Purity (By HPLC): 99.86%

### Example 7

Purification of Enzalutamide

Enzalutamide (9 gm) was charged in to the reaction flask containing methanol (7.2 ml) and heated the reaction mass to 60-65° C. to get clear solution. The obtained reaction was slowly cooled to 25-30° C. and stirred for 1 hour to obtain solid. Filtered the solid was washed with methanol. The obtained solid material was dried under vacuum at a temperature ranging from 50-60° C. DM for 4 to 5 hours to yield the pure Enzalutamide.

Yield: 6.0 gm

Chromatographic Purity (By HPLC): 99.92%

### Example 8

Purification of Enzalutamide

Enzalutamide (26 gm) was charged in to the reaction flask containing ethyl acetate (100 ml) and dried over sodium sulphate. Distilled off the solvent completely under vacuum at 50-60° C. to obtain residue. Ethyl acetate (100 ml) and n-heptane (200 ml) was slowly added to the reaction flask containing residue and stirred the reaction mass for 30 minutes to 1 hour. Filtered the material and washed with n-heptane. The obtained solid material was dried under vacuum at a temperature ranging from 50-60° C. for 4 to 5 hours to yield the pure Enzalutamide.

Yield: 22.0 gm

Chromatographic Purity (By HPLC): 99.89%

### Example 9

### Preparation of 4-bromo-2-fluoro-N-methyl benzamide (III)

N,N-Dimethylformamide (4.4 mL, 0.057 mol) was added to the 4-bromo-2-fluorobenzoic acid (175.0 g, 0.79 mol) in to reaction flask containing Ethyl acetate (1.5 L) and cooled to 15 to 20° C. Add Thionyl chloride (437.5 mL, 6.02 mol) slowly to the reaction mixture and heated to 50-55° C. for 3 to 4 hrs. The solvents were distilled out to get residue. Ethyl acetate (875 mL) was added to the residue and transferred to the addition funnel. 1.75 lit of 40% Methyl amine solution in water was taken in another flask and added 2.63 lit of Ethyl acetate and stirred at 5 to 10° C. for 30 min. Acid chloride solution was added slowly to the stirred solution at 5 to 10° C. The reaction mass was stirred at room temperature for 30 min. Conc. HCl (750.0 ml) was added to the reaction mass at 5-10° C. in 1-2 hrs and stir at room temperature for 15-20 min. The organic layer was separated and the aqueous layer was extracted twice with dichloromethane. The combined organic layers were concentrated to get residue. Water was added to the residue to obtain solid which was filtered and dried at 55-60° C. for 15-18 hrs to obtain title product.

Yield: 158.0 gm

Chromatographic Purity (By HPLC): 99.74%

### Example 10

### Preparation of 2-(3-Fluoro-4-methylcarbamoyl-phenylamino)-2-methyl-propionic acid (IV)

4-bromo-2-fluoro-N-methyl benzamide (143.0 gm, 0.61 mol) and K₂CO₃ (157.3 gm, 1.13 mol) was charged in to reaction flask containing DMF (200 mL), 1,4-dioxane (800 mL) and DM water (25.7 mL). The reaction mixture was purged with nitrogen gas for 20 min and then charged CuCl (24.4 gm, 0.25 mol), L-proline (28.6 gm, 0.25 mol) and 2-aminoisobutyric acid (157.3 gm, 1.53 mol). Continue purging nitrogen gas for 20 min followed by refluxed for 22 hrs. Solvent is distilled off completely from the obtained reaction mixture under vacuum at 70° C. The reaction mass was cooled to 10 to 15° C. and then charged DM water (286 mL). The pH of the reaction mass adjusted to 3-5 with 1 M citric acid solution to obtain the title product.

Yield: 98.0 gm

Chromatographic Purity (By HPLC): 85.37%

### Example 11

### Preparation of Enzalutamide (I)

2-(trifluoromethyl)-4-isothiocyanato benzonitrile (166.0 gm, 0.73 mol), 2-(3-Fluoro-4-methylcarbamoyl-phenylamino)-2-methyl-propionic acid (83.0 gm, 0.33 mol) were charged in to the reaction flask containing Dichloromethane (1.16 lit) and purge nitrogen for 15-20 min. Triethylamine (83.0 mL, 0.60 mol) was added at 23-28° C. and stirred for 16 hrs. The reaction mass was filtered and filtrate was distilled off to get residue. To the obtained residue Methanol (830 mL) was added and slowly added DM water and stirred at room temperature for 2-4 hrs. Filter the solid and wash with mixture of water and methanol to obtain off-white solid. The solid was purified with ethyl acetate and further with IPA and MTBE to obtain crystalline Enzalutamide.

Yield: 46.0 gm

Chromatographic Purity (By HPLC): 99.68%.

We claim:

1) An improved process for the preparation of Enzalutamide of Formula (I) 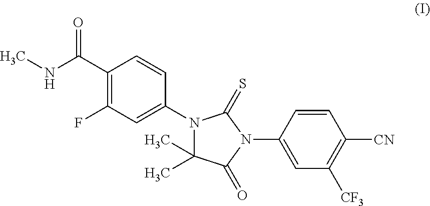 [SMILES: CNC(=O)c1ccc(N2C(=S)N(c3ccc(C#N)c(C(F)(F)F)c3)C(=O)C2(C)C)cc1F]
comprising the steps of:
a) preparation of 4-bromo-2-fluoro-N-methyl benzamide (III); 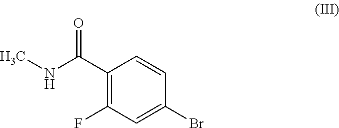
i. reacting 4-bromo-2-fluorobenzoic acid of Formula (II) 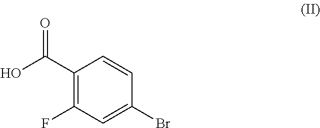 [SMILES: O=C(O)c1ccc(Br)cc1F]
with a chlorinating agent in presence of a solvent-1, followed by condensation with Methylamine in presence of a solvent-2; or
ii. reacting 4-bromo-2-fluorobenzoic acid of Formula II, with a chlorinating agent in presence of a solvent; in the ratio between 3-8 v/w times, followed by condensation with Methylamine
b) preparing 2-(3-Fluoro-4-methylcarbamoyl-phenylamino)-2-methyl-propionic acid (IV) 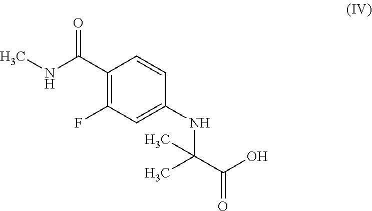 [SMILES: CNC(=O)c1ccc(NC(C)(C)C(=O)O)cc1F]
i. reacting the compound of formula (III) with 2-amino isobutyric acid in presence of amino acid and a solvent, where in the solvent is selected from a mixture of ether and an organic solvent in a ratio of 1:9 to 9:1; or
ii. reacting the compound of formula (III) with 2-amino isobutyric acid in presence of ligand and a solvent;
c) reacting the compound of Formula IV with 2-(trifluoromethyl)-4-isothiocyanato benzonitrile (V) in presence of base and a solvent to provide Enzalutamide (I); and 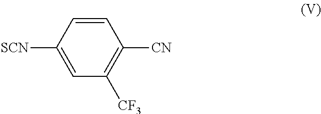
d) purifying the compound obtained in step c) further comprises of
i. providing a solution of Enzalutamide obtained in step-c) using a solvent selected from alcohol (C1-4) or Ketones (C3-6) or organic solvents (C1-8 alkanes, dimethyl formamide) or halogenated organic solvents (Methylene dichloride, Ethylene dichloride) or Ethers (Methyl tertiary butyl ether, tetrahydrofuran, Di-isopropyl ether) or sulphoxides (dimethyl sulphoxide), water or mixtures thereof;
ii. acidifying the solution using an acid selected from organic/inorganic acid not limited to formic acid, citric acid, acetic acid, Hydrochloric acid; and
iii. isolating the substantially pure Enzalutamide having a purity of greater than 99.5%.

2) A process for the preparation of Enzalutamide according to claim 1, wherein chlorinating agent is selected from Oxalyl chloride, Thionyl chloride, PCl₃, PCl₅, POCl₃.

3) A process for the preparation of Enzalutamide according to claim 1, wherein solvent-1 in step a) is selected from solvent selected from halogenated hydrocarbons such as methylene dichloride, ethylene dichloride, chloroform; esters such as ethyl acetate, isopropyl acetate, isobutyl acetate, methyl acetate; alcohols such as C1-5 alcohols; sulfoxides such as dimethylsulfoxide; aromatic hydrocarbons such as toluene, xylene; ketones such as acetone, methyl ethyl ketone, methyl isobutyl ketone; water or mixtures thereof. or mixtures thereof; Solvent-2 in step a) is selected from ethers such as Methyl tert-butyl ether, Tetrahydrofuran, Methoxyethane, 2-(2-Methoxyethoxy)ethanol, Di-tert-butyl ether, Diethyl ether, Di-ethylene glycol diethyl ether, Diglyme, Di-isopropyl ether, Dimethoxymethane, 1,4-Dioxane, 1,3-dioxane, 1,2-dimethoxy ethane, Ethyl tert-butyl ether, 2-Methyl tetrahydrofuran, Morpholine; Glycol ethers such as 2-Butoxyethanol, Diglyme, Dimethoxyethane, 2-Ethoxy ethanol, 2-(2-Ethoxyethoxy)ethanol, 2-Methoxyethanol, 2-(2-Methoxyethoxy) ethanol, Octaethylene glycol monododecyl ether, Pentaethylene glycol monododecyl ether, Phenoxyethanol, Propylene glycol methyl ether acetate, Tetraethylene glycol dimethyl ether, Triethylene glycol, Triethylene glycol dimethyl ether; water or mixtures thereof; Solvent in step a) is selected from halogenated hydrocarbons such as methylene dichloride, ethylene dichloride, chloroform or mixtures thereof, in the ratio between 3-8 v/w times.

4) A process for the preparation of Enzalutamide according to claim 1, wherein ligand in step b) is selected from 2-acetyl cyclohexanone; N,N-dimethyl glycine.HCl, cyclic amino acid such as Proline, L-Proline, D-Proline, Hydroxyproline, Pseudoproline, 1-Aminocyclopropane-1-carboxylic acid, Azetidine-2-carboxylic acid.

5) A process for the preparation of Enzalutamide according to claim 1, wherein solvent in step b) is selected from alcohols such as ethanol, ethylene glycols, n-butanol, isopropanol; ether such as tetrahydrofuran, 1,4-dioxane, 1,3-dioxane, 1,2-dimethoxy ethane and an organic solvent such as acetonitrile, dimethylformamide dimethylsulfoxide in a ratio of 1:9 to 9:1

6) A process for the preparation of Enzalutamide according to claim 1, wherein base is selected from inorganic or organic bases, such as triethylamine, diisoproyl ethylamine, tributyl amine, N,N-dimethyl aniline, pyridine, N-methyl morpholine, DBN, DBU.

7) A process for the preparation of Enzalutamide according to claim 1, wherein solvent in step c) selected from ketone solvents such as acetone, methyl isobutyl ketone;
acetonitrile or mixtures.

8) A process for the preparation of Enzalutamide (I) comprising the steps of:
a) preparation of 4-bromo-2-fluoro-N-methyl benzamide (III); 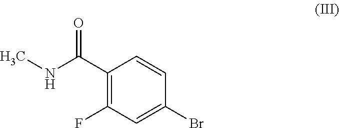
i. reacting 4-bromo-2-fluorobenzoic acid of Formula (II) 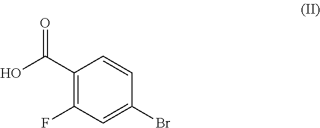 [SMILES: O=C(O)c1ccc(Br)cc1F]
with a chlorinating agent in presence of a solvent-1, followed by condensation with Methylamine in presence of a solvent-2; or
ii. reacting 4-bromo-2-fluorobenzoic acid of Formula II, with a chlorinating agent in presence of a solvent; in the ratio between 3-8 v/w times, followed by condensation with Methylamine
b) preparing 2-(3-Fluoro-4-methylcarbamoyl-phenylamino)-2-methyl-propionic acid (IV) 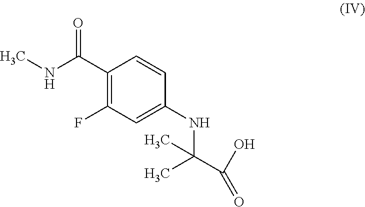 [SMILES: CNC(=O)c1ccc(NC(C)(C)C(=O)O)cc1F]
i. reacting the compound of formula (III) with 2-amino isobutyric acid in presence of amino acid and a solvent, where in the solvent is selected from a mixture of ether and an organic solvent in a ratio of 1:9 to 9:1; or
ii. reacting the compound of formula (III) with 2-amino isobutyric acid in presence of ligand and a solvent;
c) converting 2-(3-Fluoro-4-methylcarbamoyl-phenylamino)-2-methyl-propionic acid of Formula IV to Enzalutamide (I)

9) A process for the preparation of Enzalutamide intermediate according to claim 8, wherein chlorinating agent is selected from Oxalyl chloride, Thionyl chloride, PCl₃, PCl₅, POCl₃ and the solvent in step a) is selected from halogenated hydrocarbons such as methylene dichloride, ethylene dichloride, chloroform or mixtures thereof.

10) A process for the preparation of Enzalutamide intermediate according to claim 8, wherein Ligand is selected from 2-acetyl cyclohexanone, N,N-dimethyl glycine.HCl and solvent in step b) selected from alcohols, such as ethanol, ethylene glycols, n-butanol, isopropanol; ethers such as tetrahydrofuran, 1,4-dioxane, 1,3-dioxane, 1,2-dimethoxy ethane; aprotic polar solvents like acetonitrile, dimethylformamide, dimethylsulfoxide or mixtures thereof.

11) Substantially pure crystalline Enzalutamide having XRPD pattern comprising at least 7 characteristic peaks possessing peaks selected from 6.5, 9.8, 13.1, 15.8, 16.0, 16.7, 18.9, 19.5, 19.7, 21.2, 22.6, 25.5±0.2°2θ.

12) Substantially pure crystalline Enzalutamide according to claim further characterized by XRPD peaks at 12.3, 13.5, 14.3, 15.0, 17.4, 21.8, 24.4±0.2°2θ.

13) Substantially pure crystalline Enzalutamide according to claim 11 characterized by X-ray powder diffraction pattern substantially according to FIG. 1.
